(19) United States
(12) Patent Application Publication
(10) Pub. No.: US 2025/0221952 A1
(43) Pub. Date: Jul. 10, 2025

(54) METHODS AND COMPOSITIONS FOR LARGE MODIFICATIONS OF ATP OUTPUT BY INNER MITOCHONDRIAL MEMBRANE LATCHING

(71) Applicant: Louis Dischler, Spartanburg, SC (US)

(72) Inventor: Louis Dischler, Spartanburg, SC (US)

(21) Appl. No.: 19/080,598

(22) Filed: Mar. 14, 2025

(51) Int. Cl. A61K 31/198; A61K 31/19; A61K 31/194; A61K 31/352; A61K 31/4406; A61K 31/4745

(52) U.S. Cl. CPC A61K 31/198; A61K 31/19; A61K 31/194; A61K 31/352; A61K 31/4406; A61K 31/4745

(57) ABSTRACT

Disclosed are methods and antiaging compositions for the rapid restoration of mitochondrial health by demethylation, the dramatic modification of ATP output by up-latching or down-latching of the inner mitochondrial membrane, and the rapid elimination of symptoms due to mitochondrial dysfunction.

The disclosed methods and antiaging compositions primarily relate to the rapid restoration of mitochondrial health by demethylation, the surprisingly large modifications of ATP output by up-latching or down-latching of the inner mitochondrial membrane, and the rapid elimination of symptoms due to mitochondrial dysfunction.

### BACKGROUND OF THE INVENTION

Mitochondria are energy producing organelles with their own DNA (mtDNA). They feature two phospholipid bilayer membranes, the outer mitochondrial membrane (OMM) and the inner mitochondrial membrane (IMM). The IMM encloses a matrix containing mtDNA, and has five complexes for oxidative phosphorylation (OXPHOS) embedded. A proton gradient across the IMM is created by complexes that pump protons from the matrix into the intermembrane space. The return flow of protons through another embedded IMM complex-ATP synthase-produces adenosine triphosphate (ATP). The energetic decline of mitochondria is a known aspect and driver of aging. It has been estimated that ATP output declines by some 8% per decade. Numerous diseases and syndromes have been associated with and made worse by ATP insufficiency.

Insufficiency is in large part driven by mtDNA mutations and epimutations (methylation). In most cases, mitochondrial quality control (QC) can deal with mutations via PINK1/Parkin proteins that label mitochondria with zero surface potential, but responds poorly against epimutations, as methylation may turn down the production of IMM complexes for OXPHOS, thereby lowering surface potential but not sending it to zero. Methods and supplements for reducing mtDNA methylation and increasing the complexes in the IMM would thus improve mitochondrial energetics (the generation of adenosine triphosphate), and that would enhance athletic performance, improve organ health, and reduce the severity of many diseases of aging.

The aging of mtDNA proceeds in rough parallel to that of nDNA. In nDNA, methylation errors degrade the epigenome, while in mtDNA, methylation can disturb the correct balance of OXPHOS complexes and reduce ATP output. There is no known mtDNA epigenome as such, so all methylation can be seen as epimutations and thus undesirable. Most genes coding for mitochondria are located in the nDNA, but of the 37 genes encoded by human mtDNA, 13 provide instructions for making protein subunits for OXPHOS. If just one mutated gene fails to function correctly, then oxidative phosphorylation cannot be supported by that mtDNA loop when isolated in a fissioned mitochondria. The IMM membrane potential (ΔΨm) thus falls to zero, allowing such mitochondria to be labeled by the PINK1/Parkin QC process that ultimately results in its degradation in lysosomes (mitophagy). Undesirable methylation of mtDNA also increases with age and exposure to certain viruses and drugs, but there is no QC system to eliminate them, as fissioned mitochondria with a single molecule of methylated mtDNA that produces a lower but nonzero membrane potential can escape mitophagy. With lower rates of ATP production producing lower levels of reactive oxygen species (ROS), methylated mtDNA may even have a survival advantage.

The present disclosure provides rapid solutions to the problems of ATP insufficiency and mtDNA methylation.

This application claims benefit from provisional Application Nos. 63/640,071 filed Apr. 29, 2024; and 63/676,388, filed Jul. 28, 2024. This application is also related to applicant's U.S. Pat. Nos. 11,541,022, filed May 2, 2022; U.S. Pat. No. 11,504,351, filed Dec. 7, 2021; U.S. Pat. No. 11,344,528, filed Feb. 16, 2021; U.S. Pat. No. 11,324,764, filed Jul. 20, 2021; U.S. Pat. No. 11,317,646, filed May 3, 2022; and U.S. Pat. No. 11,090,320, filed Feb. 16, 2021; with the contents of each of the above-listed publications hereby incorporated by reference in their entireties, for all purposes.

In those previous patents, compositions and methods were disclosed for removing undesirable methylation. This included a method for alternating mitochondrial fission and fusion over a period of weeks. The present disclosure will show how methylation can be eliminated in as little as 2 hours, with no need of mitochondrial fission or fusion, increasing mitochondrial energetics substantially. It will also show that far greater increases in energetics can be achieved by up-latching the IMM, which can also be employed during the systemic repopulation of stem cell niches with a UCP2 blocker.

### ABBREVIATIONS

The following abbreviations are used herein:
AHA Alpha hydroxycarboxylic acid
AKG Alpha-ketoglutarate
AAKG Arginine-alpha-ketoglutarate
ATP Adenosine triphosphate
B3 Nicotinic acid or nicotinamide
DHM Dihydromyricetin
IMM Inner mitochondrial membrane
MCT Medium chain triglycerides
mtDNA Mitochondrial DNA
NAD Nicotinamide adenine dinucleotide
NAM Nicotinamide
NAM+R Nicotinamide plus ribose
nDNA Nuclear DNA
OMM Outer mitochondrial membrane
PQQ Pyrroloquinoline quinone
QC Mitochondrial quality control, PINK1/Parkin
ROS Reactive oxygen species
SC Stem cells
TET Enzymes that remove methyl groups
UCP2 Uncoupling protein 2
ΔΨm Mitochondrial membrane potential

### SUMMARY OF THE INVENTION

The primary source of aging is herein proposed to be a combination of mutations and incorrect methylation of mitochondrial and nuclear DNA, called epimutations. Epimutations of the nuclear epigenome results in ever increasing epigenetic age. For mitochondrial DNA (mtDNA), there is no known epigenome, thus any methylation of mtDNA can be seen as an epimutation. Such epimutations produce an incorrect mix of OXPHOS complexes, and generally lower mitochondrial energetics.

As the OXPHOS complexes expressed in the IMM drive ATP output, methods and supplements for increasing these complexes in the IMM would increase ATP output and athletic performance, improve organ health, and cure or reduce the severity of many diseases of aging.

In addition, methods and supplements for decreasing OXPHOS complexes in the IMM will decrease ATP output and concomitant ROS production, which can be useful for controlling diseases as diverse as plaque psoriasis and Multiple Sclerosis (MS), and for eliminating defective OXPHOS complexes from the IMM.

Upregulating and downregulating of mitochondrial IMM complexes can be used independently, or in alternating fashion by up-latching and down-latching of IMM complexes, herein referred to simply as up-latching and down-latching.

The disclosed protocols and antiaging supplements provide for removing methylation marks from mtDNA in as little as one treatment, thus restoring ATP output rapidly, and provides for further increasing ATP output by IMM up-latching, which can also be achieved rapidly.

Mitochondria have several morphological and functional states that are useful in the present invention. These are:
1. Morphology—fission and fusion
2. Quiescence—on and off
3. IMM latching—up and down

Fission and fusion are well known morphological states, wherein fission is necessary for PINK1/Parkin quality control and apoptosis of senescent cells, while fusion is helpful in mixing mitochondrial components and promoting symmetric division of stem cells. Stem cells are normally quiescent due to numerous UCP2 uncoupling pores that allow protons to bypass ATP-synthase in the IMM. Blocking those pores with the fullerene C60—a putative UCP2 pore blocker that Applicant disclosed in his related patents—banishes quiescence and promotes stem cell division. With stem cell mitochondria in the fusion state, symmetric division is promoted over asymmetric division.

Nonlimiting examples of mitochondrial fusion promoters are stearic acid and/or sources thereof such as monosodium stearate; myricetin, dihydromyricetin (DHM) and other derivatives and analogs of myricetin; sulforaphane and/or sources thereof.

Applicant has discovered another mitochondrial state, herein called IMM latching, in which ATP output is increased by plasticizing the IMM, allowing it to become super-saturated with complexes for oxidative phosphorylation. ATP output can be latched at a level many times higher than baseline, a state that can be substantially maintained for some time after the plasticizer is gone. This increase in ATP output can be measured by the simple proxy of a repetitive exercise routine, such as bicep curls to failure.

IMM latching is believed to result in a static but metastable mitochondrial morphology, as changing the morphology with either fission or fusion supplements can immediately collapse ATP output to a lower level.

An optional first step in maximizing ATP output is the removal of mtDNA methylation. A useful antiaging composition for removing aberrant methylation of mtDNA comprises a mtDNA biogenesis promoter and a demethylase (TET enzyme) promoter. By supplying a sufficiently high dosage of the biogenesis promoter, all or most mtDNA are forced into division simultaneously, and can be demethylated with a small number of doses, even as few as one. An exemplary treatment would comprise at least about 50 mg of pyrroloquinoline quinone (PQQ), or salts, esters and other derivatives thereof, and at least 250 mg of alpha-ketoglutarate (AKG) or salt thereof. At least 75 mg PQQ or derivatives thereof and at least 500 mg AKG or salt thereof are preferred, and at least 100 mg PQQ or derivatives thereof and about 1 g AKG or more are most preferred for treatment with a single dose. Combined doses are preferably delivered by oral means. As nonlimiting examples, delivery can be made by tablet, capsule or powders dissolved or dispersed in liquids.

Nonlimiting examples of TET enzyme promoters useful in the instant invention include alpha-ketoglutaric acid (AKA), and its salt, alpha-ketoglutarate. Useful salts of AKA include ammonium alpha-ketoglutarate, arginine alpha-ketoglutarate (AAKG), calcium alpha-ketoglutarate, creatine alpha-ketoglutarate, glutamine alpha-ketoglutarate, leucine alpha-ketoglutarate, lithium alpha-ketoglutarate, lysine ketoglutarate, magnesium alpha-ketoglutarate, ornithine alpha-ketoglutarate, potassium alpha-ketoglutarate, sodium alpha-ketoglutarate, and taurine alpha-ketoglutarate. Demethylase activity depends in part on the availability of alpha-ketoglutarate, which is an intermediate in the Krebs cycle.

It is thus a principle object of some aspects of the present invention to eliminate aberrant epigenetic marks on mammalian mtDNA, thereby increasing ATP output.

Another object of some embodiments of the present invention is to modify ATP output by a previously unknown phenomenon herein called inner mitochondrial membrane (IMM) latching. Protocols and supplements are disclosed for up-latching or down-latching of the IMM, thereby increasing or decreasing ATP output, respectively.

It was found that supplementation with certain carboxylic acids or salts thereof (up-latchers) can be used in conjunction with enhanced mtDNA count to increase ATP output to a level many times that of baseline. It is believed that up-latchers function as IMM plasticizers, allowing OXPHOS complexes to enter the IMM at concentrations greater than normal, where they can remain for hours or days without further supplementation.

Of the two mitochondrial membranes, the outer OMM is lipid rich while the inner IMM is protein rich. The IMM proteome (all the protein components) has been estimated at 70% of the IMM in its normal, untreated state. While this is very high, it can be forced higher by suitable plasticizers. After treatment with plasticizers, this higher than normal concentration of OXPHOS complexes appear to be latched in place, much like the ratcheting mechanism of a socket wrench allows motion in one direction while preventing motion in the opposite direction. The analogous ratcheting pawl in mitochondria is believed to be the compressive force of the IMM that would otherwise prevent more complexes from entering, while in the latched, supersaturated state it prevents them from leaving. With a sufficient supply of OXPHOS complexes in the matrix, treatments with up-latching plasticizers can be repeated to produce latching at even higher levels. This is herein termed “IMM ratcheting,” or more simply, “ratcheting.”

Latching and ratcheting may be promoted by oral supplementation of certain carboxylic acids or salts thereof, herein termed “up-latchers.” Salts are preferred, and salts of calcium, magnesium, potassium and sodium are most preferred for up-latchers. Different salts may be used together. Salts of amino acids may be used, such as in lysine lactate.

Alpha-ketoglutarate (AKG), itself a weak up-latcher, synergistically increases the effectiveness of latching in combination with other up-latchers, and does double duty by promoting demethylase for removing epimutations. AKG can thus be effectively combined with a biogenesis promoter and other up-latchers. Preferred biogenesis promoters include pyrroloquinoline quinone (PQQ), its esters, isomers, and derivatives thereof.

Rapid up-latchers include carboxylic acids with a single carbon backbone having a first carboxyl end group and a hydroxy group at the alpha position relative to the first carboxyl end group, and/or a second carboxyl end group. These are alpha-hydroxy acids (AHAs) and dicarboxylic acids. Slow up-latchers include lycopene and the structurally similar colorless carotenoids, phytoene and phytofluene. All three carotenoids are distinguished by the lack of bulky end groups, with lycopene preferred.

A plurality of up-latchers can be used together for synergistically enhanced ATP output. It is likely that the 200 cell types in the body have varying uptakes of up-latchers—most particularly in the brain with its blood-brain barrier—and up-latchers may have different plasticizing efficiencies near the various complexes in the IMM. In fact, a combination of different up-latchers can produce much higher levels of ATP output, as measured herein by the proxy of bicep curls to failure.

Thus another principle object of some aspects of the present invention is latching ATP output at a higher level by increasing the oxidative phosphorylation complexes in the IMM to a supersaturated level with one—or preferably a plurality—of up-latchers.

Another principle object of some aspects of the present invention is the ratcheting up of ATP output with sequential dosing of up-latchers to achieve even higher levels of ATP output.

Another principle object of some aspects of the present invention is the combination of alpha-ketoglutarate with other up-latchers to achieve higher levels of ATP output.

Another principle object of some aspects of the present invention is the combination of latching supplements with biogenesis promoters.

Another principle object of some aspects of the present invention is the combination of latching supplements with a UCP2 blocker.

Alpha hydroxycarboxylic acids are carboxylic acids comprising a hydroxy group located one carbon from the acid group (the alpha position). Up-latchers should preferably have a relatively straight carbon backbone with one or two carboxyl end groups (COOH). The dicarboxylic fumaric acid comprises two carboxyl end groups (COOH), and appears to be more efficient than the AHAs evaluated. Dosing in combination with other up-latchers provides latching that is substantially more than additive.

While proxy ATP evidence suggests that alpha hydroxycarboxylic acids and dicarboxylic acids plasticize the inner mitochondrial membrane, producing up-latching of ATP output, it was found that many other plasticizers have the opposite effect—reversing the latching action and allowing complexes to escape the IMM. This is herein called down-latching. As examples, lactate is nominally an up-latcher, but the lactate esters ethyl lactate and butyl lactate are strong down-latchers. Glycerol, a sugar alcohol, is a strong down-latcher. Triethanolamine (TEA) is a strong down-latcher, as are the phthalates used in plastic products that can transfer into food. Supplements that modify mitochondrial morphology can also promote down-latching. These include fission and fusion promoters, though fusion promoters can be used before or concomitantly with up-latchers. As the latched condition is metastable, changing up-latched IMM morphology can result in its partial or complete collapse, especially with fission.

Up-latching and down-latching provide the ability to rapidly increase or decrease ATP output as desired, and when used in an alternating fashion, can remove and replace embedded IMM complexes that have become nonfunctional or dysfunctional.

Thus another object of some embodiments of the present invention is to expel and replace defective complexes from the IMM by alternating up-latching with down-latching.

Another object of some embodiments of the present invention is to ameliorate metabolic deficiencies that result from drugs, disease, or aging.

Another object of some embodiments of the present invention is to increase mitochondrial ATP output for exercise and sporting activities.

Another object of some embodiments of the present invention is to increase mitochondrial ATP output prior to or during SC proliferation.

Another object of some embodiments of the present invention is to decrease mitochondrial ATP output with down-latching for treating syndromes associated at least in part with ATP oversufficiency.

Another object of some embodiments of the present invention is to decrease mitochondrial ATP output to reduce ROS.

In one embodiment, a dose of PQQ or derivative thereof is combined with AKG or salt thereof to increase mtDNA count and simultaneously eliminate methylation.

In another embodiment, the OXPHOS component count in the inner mitochondrial membrane is increased, thereby increasing ATP output.

In one embodiment, the surface area of the inner mitochondrial membrane is increased, thereby allowing more OXPHOS components to enter, increasing ATP output.

In another embodiment, the OXPHOS components of the inner mitochondrial membrane is increased in steps, up-ratcheting ATP output.

In another embodiment, mitochondrial up-latching is combined with a UCP2 blocker to enhance the symmetric proliferation of stem cells.

In another embodiment, supplements for mitochondrial biogenesis and demethylation are combined to banish symptoms of multiple sclerosis and diseases with similar etiologies.

Additional objects, advantages and novel features of the invention will be set forth in part in the description which follows, and in part will become apparent to those skilled in the art upon examination of the following disclosure, or may be learned by practice of the invention. The objects and advantages of the invention may be realized and attained by means of the instrumentalities and combinations particularly pointed out in the appended claims.

These together with other objects of the invention and various novel features that characterize the invention are particularized in the claims that form part of this disclosure. For a better understanding of the invention, its advantages and the specific objects attained by its uses, reference should be made to the accompanying drawings and descriptive matter in which preferred embodiments of the invention are illustrated.

### BRIEF DESCRIPTION OF THE DRAWINGS

The protocols and nutritive compositions will be better understood and objects other than those set forth above will become apparent when consideration is given to the following detailed descriptions thereof. Such descriptions reference the annexed drawings, wherein:

FIG. 1 is a plot of ATP output (as represented by the proxy of bicep curls to failure) vs time, when dosing with various up-latchers used singly and in combination, illustrating the combinational synergy in boosting ATP output with multiple plasticizers.

FIG. 2 is a plot of ATP output (as represented by the proxy of bicep curls to failure) vs time, for dosing with equal amounts of nine combined up-latchers at four dosage levels.

FIG. 3A is a plot of the partial replication of a trial of a previous protocol to repair epigenetic damage to mtDNA, followed by an investigation that discovered up-latching and down-latching. FIG. 3B is a plot of the previous protocol to repair epigenetic damage to mtDNA.

FIG. 4 shows the effect of dosing with the down-latcher glycerol.

FIG. 5 shows the effect of dosing with down-latchers followed by dosing with up-latchers.

FIG. 6 shows the effect of dosing with 2 up-latchers, a UCP2 blocker, and a down-latcher.

FIG. 7 compares an up-latching carotenoid with a non-latching carotenoid.

FIG. 8 shows up-latching with a carotenoid, followed by up-ratcheting with the salt of a dicarboxylic acid.

FIG. 9 illustrates the anomalous behaviors of the up-latcher lactate and the fusion agent DHM.

FIG. 10 is a plot of bicep curl repetitions to failure at 15 minute intervals after dosing with the non-latcher pyruvic acid, neutralized to sodium pyruvate.

FIG. 11 is a plot of bicep curls repetitions to failure after dosing with the down-latcher citric acid, neutralized to sodium/potassium citrate.

### DETAILED DESCRIPTION OF THE INVENTION

Herein a new and unexpected mitochondrial state is reported, viz, inner mitochondrial membrane (IMM) latching. Latching may take the form of up-latching or down-latching, wherein plasticizers are used to increase or decrease the concentration of OXPHOS complexes present in the IMM, thus increasing or decreasing ATP output.

The various mitochondrial states can be combined to achieve specific ends for health and longevity. These states are up-latching/down-latching, fission/fusion, and stem cell (SC) quiescence.

For example:
I. Up-latching with fusion for higher ATP output, and for promoting SC symmetric proliferation when used with a UCP2 blocker for banishing quiescence.
II. Up-latching with fission for higher ATP output while promoting senescent cell apoptosis.
III. Down-latching with fusion for reducing inflammation.
IV. Down-latching with fission for promoting mito QC.

In addition, biogenesis can be combined with demethylase to achieve an essentially complete removal of mtDNA methylation. It is preferably achieved, for some individuals, without fission promoters.

ATP decline during aging is often due to epigenetic alteration of mtDNA by methyl groups. Such damage cannot be detected by mitochondrial QC as membrane potential typically does not go to zero. Methylation damage can build over time as the presence of methyltransferase in mitochondria maintains it, while QC does not remove it. By promoting demethylase and concomitantly promoting replication of mtDNA, epigenetic damage can be eliminated with one treatment, even without supplements for fusion or fission.

Methylation of mtDNA can result in reduced ATP output and fatigue, and can also contribute to serious and debilitating conditions. For instance, it is known that statins can inhibit mitochondrial complex III, which deficiency can then promote the hypermethylation of mitochondrial genes via the buildup of 2-hydroxyglutarate, which inhibits demethylase. Hypermethylation can then result in an MS-like pathology. Given the similarity of these symptoms to multiple sclerosis, it is herein suggested that mitochondrial demethylation via dosing of PQQ and AKG is a viable treatment for MS and similar pathologies. Antibiotics like fluoroquinolone can likewise methylate mtDNA.

In those suffering from age-related ATP decline, energetics can be rapidly restored to a more youthful level by plasticization of the IMM with up-latchers. It might be expected that ATP output could be increased by increasing the supply of OXPHOS complexes in the mitochondrial matrix with a biogenesis promoter. However, increased OXPHOS complexes in the matrix cannot increase ATP until they are incorporated into the IMM, and the IMM appears to have a saturation limit.

The putative saturation limit can be bypassed by the present invention to produce supersaturated and unsaturated states. By the appropriate choice of plasticizers, OXPHOS complex density can be increased or decreased in the IMM, increasing and decreasing ATP output as desired. IMM plasticization with the salts of specific carboxylic acids is hypothesized to allow a greater packing density of complexes, even after the carboxylic plasticizer disappears, while certain other plasticizers do the reverse. While not desiring to be limited by theory, it is believed that ATP output is a function of the inner mitochondrial membrane surface area times its embedded complex density. Up-latchers increase complex density, and the increased density results in increased ATP output, while the IMM surface area either remains the same, or is more likely increased by a homeostatic mechanism keeping complexes separated, thus producing more surface area for latching at a higher level, i.e., ratcheting. The combination of multiple carboxylic up-latchers was found to produce very large improvements in exercise stamina (as measured by bicep curl reps-to-failure) that are surprisingly persistent.

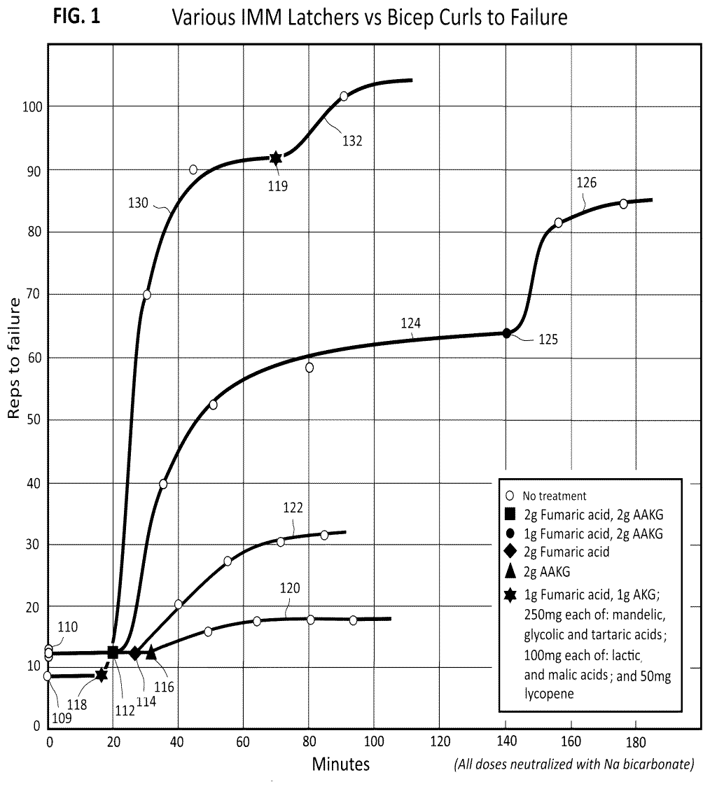

Referring now to the drawings wherein like numerals refer to like parts, FIG. 1 shows a plot of reps-to-failure (bicep curls to failure, herein a proxy for ATP output) vs minutes. In this figure, the lifted weight was a 15 pound dumbbell. Filled data points represent reps-to-failure measured immediately before treatment, while open circles represent reps-to-failure without treatment. The plot shows four experiments on different days, all with acids neutralized with sodium bicarbonate. Three began at a baseline of 12 reps-to-failure indicated by the 3 overlapping open circles 110, while the fourth began with a slightly lower baseline of 9 reps-to-failure, indicated by open circle 109. The filled square 112 represents a dose of fumaric acid and AAKG, producing curve 124 that levels off at point 125 with 64 reps-to-failure—a more than 5-fold improvement over starting point 112. At point 125, another fumaric acid/AAKG dose resulted in a ratcheting up in reps-to-failure, with the reps-to-failure curve 126 reaching a 7-fold improvement of baseline. Curve 122 is a plot of fumaric acid dosed at point 114, and curve 120 is a plot of AAKG dosed at point 116. Both are much lower than curve segment 124, indicating that the combination of neutralized fumaric acid (e.g., sodium fumarate) and AAKG is synergistic and greater than might be achieved by a simple sum of the individual increases. Eight different up-latchers were dosed at point 118 producing even higher reps-to-failure, indicated by curve segment 130, which reached a 10-fold increase at point 119 over its 9-rep baseline between points 109 and 118. The same 8 up-latchers were dosed again at point 119, with reps-to-failure reaching an 11-fold increase as represented by curve segment 132. The combination of 8 up-latchers also produced a much faster response.

Such large increases of ATP output have heretofore been unachievable. For comparison, methylene blue has been promoted for increasing ATP, but this was not found to be the case, at least with reps-to-failure. A 100 mg dose produced no change from baseline of 11 reps at 15 minutes, and no change at one hour.

In separate trials, results for neutralized malic acid were nearly identical to that shown for neutralized fumaric acid in FIG. 1. Malic and fumaric acids will be converted in the body to malate and fumarate respectively, if not neutralized beforehand, and anions appear to be responsible for up-latching in all cases except for carotenoid up-latchers.

In another trial, succinic acid appeared to be an indirect up-latcher. Reps-to-failure peaked at about half that of fumaric and/or malic acids when combined with AAKG and a neutralizing base, and were delayed by about 15 minutes. It is known that succinate is converted into fumarate by the enzyme succinate dehydrogenase, and this may explain the delay, in which case fumarate is the actual up-latcher.

In yet another trial, 1 gram of another dicarboxylic acid, adipic acid, raised reps-to-failure from 11 to 31 in 20 minutes when combined with 1 gram of AAKG and sodium bicarbonate as a neutralizer.

Fast up-latchers comprise carboxylic acids with a single carbon backbone having a first carboxyl end group and a hydroxy group at the alpha position relative to the first carboxyl end group, and/or a second carboxyl end group. Slow up-latchers comprise the carotenoids lycopene and the similar but colorless carotenoids, phytoene and phytofluene, all three of which have open-chain end groups of their polyene backbones, distinguishing them from non-latching carotenoids that have terminal rings. Up-latching carboxylic acids and carotenoids should have no more than one terminal ring. Mandelic acid is an example of a carboxylic (AHA) up-latcher with one terminal ring.

In most trials the dose acidity was substantially neutralized with sodium bicarbonate, while in some cases a mixture of sodium and potassium bicarbonates was used. Sodium potassium, calcium, and magnesium carbonates, bicarbonates, oxides, hydroxides, or other well-known neutralizers may be used.

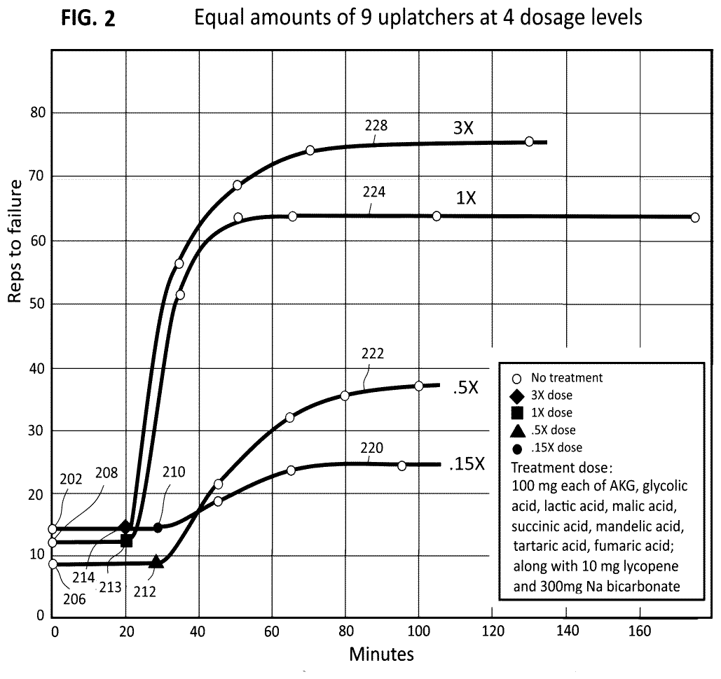

Turning now to FIG. 2, A dose of 9 up-latchers was trialed at 4 dose levels. A combined dose of 810 mg (X) was dosed at 0.15X at point 210, 0.5X at point 212, 1X at point 213, and 3X at point 214. The baseline for 0.15X and 3X was 15 reps at point 202, the baseline for 1X was 13 reps at point 208, and the baseline for .5X was 9 reps at point 206. All 4 doses behaved similarly, however, reps-to-failure occurred much faster with higher doses, and the 1X dose at the upper limit indicated by curve 224 was only 10 reps below the upper limit for the 3X dose indicated by curve 228. The upper limit curve 222 for .5X and the upper limit curve 220 for the .15X treatments were much lower.

The combined 1X up-latcher dose of 810 mg that reached the 60 reps-to-failure curve 224 in FIG. 2 was just ⅕ the combined 2 component dose in FIG. 1 that reached approximately the same reps-to-failure curve 124, illustrating again the advantage of using mixed up-latchers.

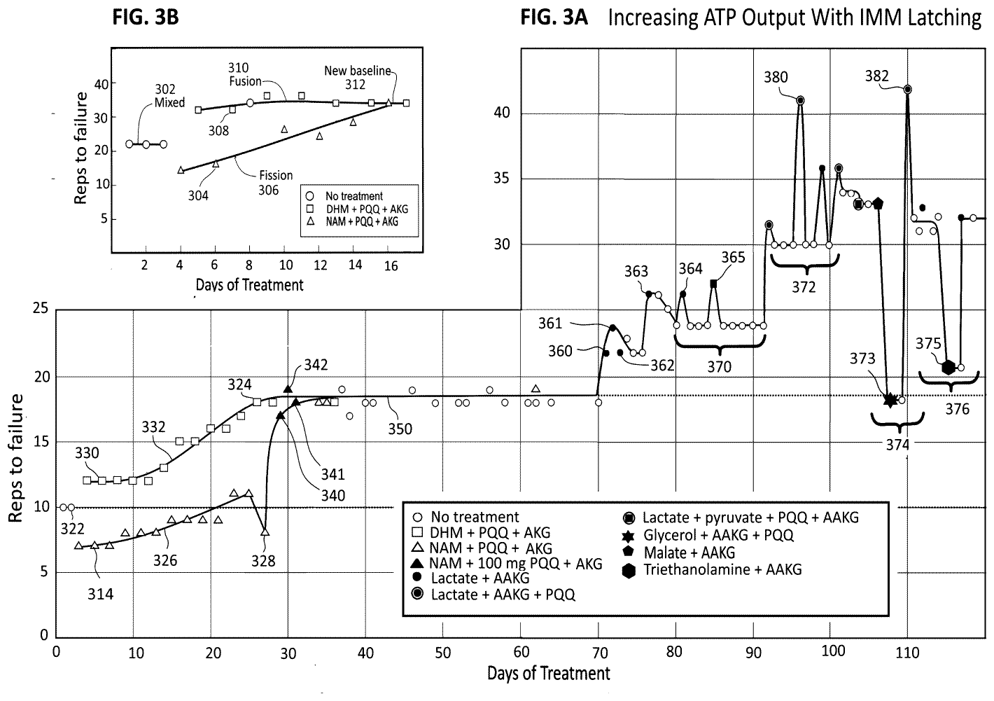

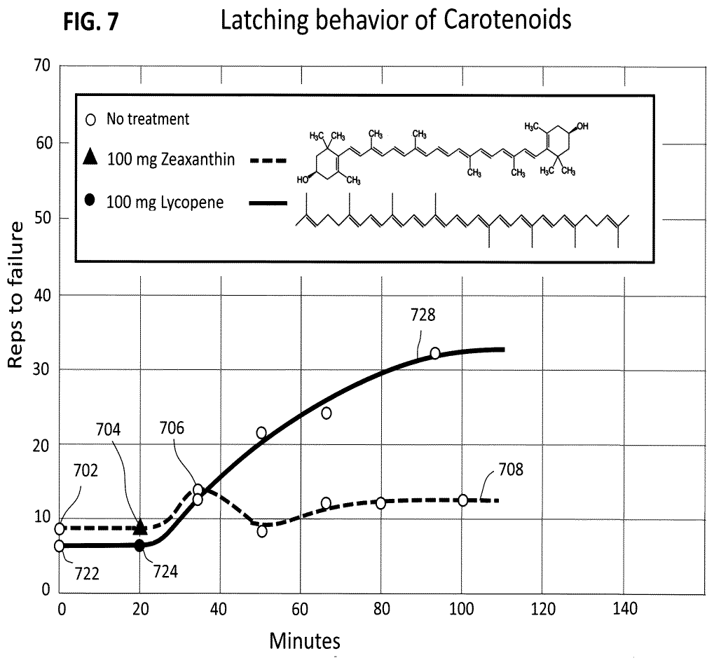

Turning now to FIG. 3A, latching and ratcheting were first discovered in an attempt to reproduce applicant's patented fission/fusion process for removing methylation of mtDNA, this time with reduced dosages. The plot from the original experiment (U.S. Pat. No. 11,324,764, FIG. 7) is shown in FIG. 3B. This was a trial by a 68 year old individual who suffered statin damage. In that trial, fission (with niacinamide-NAM) and fusion (with dihydromyricetin-DHM) were administered on alternate days as unitary doses that also contained 20 mg PQQ and 1 g AKG. All treatment points were tested 2 hours after treatment, and all tests were at least 24 hours apart. The un-treated initial state (mixed fission and fusion) is indicated by line 302, while the fission curve 306 is lower, and the fusion curve 310 is higher. Fission treatments are indicated by triangles such as 304 and fusion treatments by squares such as 308. This produced a bifurcated curve that merged to create a new baseline 312, after which there was no difference between fission and fusion.

Alternating fission and fusion produces a bifurcated curve because mtDNA damage is exposed during fission but hidden during fusion. Damage is generally epigenetic rather than genetic, as QC can eliminate mutation errors via mitophagy, but not most epimutations. Though in some syndromes this is not true, for example, in Parkinson's disease.

In FIG. 3A, the plot shows the result of a trial of a 72 year old individual suffering from damage to mitochondria after a case of shingles (reactivation of the varicella-zoster virus). Fission and fusion supplements were administered on alternate days as unitary doses that also contained 20 mg PQQ and one gram of AAKG, much as in the previous trial reproduced in FIG. 3B. In the new experiment shown in FIG. 3A, fusion treatments are again represented by squares 330, and fission treatments by triangles 314, producing a bifurcated curve with the fusion curve 332 above the untreated mixed state 322, and a fission curve 326 below both. For this new experiment, dosage levels of promoters of fusion and fission were reduced by ⅔ and ½, respectively. These reduced levels proved to be inadequate in achieving a rapid elimination of methylation. While the bifurcation was similar to the original experiment, the fusion curve 332 was considerably lower and the fission curve 326 was rising much more slowly than in FIG. 3B. Dosages were therefore increased. At point 324, the fusion dose was tripled, but as the fusion curve 332 had already risen to the new baseline 350, no significant change was noted. The dose of the fission agent was doubled at point 328, which reduced the reps-to-failure immediately, indicating that complete fission was not being obtained with the lower dose. The major surprise was at point 340, where the PQQ dose was increased 5-fold to 100 mg. This resulted in the near instant merger of fission and fusion lines, indicating that the higher dosage of PQQ was key to rapid elimination of methylation. It is believed that placing all or nearly all mtDNA into division allowed the AAKG dose, which is a promoter of TET demethylase enzymes, to eliminate methylation in one pass. With the higher PQQ dose, fission and fusion are not needed at all, if only epimutations (as opposed to mutations) are to be removed. Subsequent treatments 341, 342 with this higher dose of PQQ increased reps-to-failure only slightly, with a new baseline 350.

Eliminating epigenetic damage with just one dose has great benefit in reversing mitochondria linked diseases. For example in MS, which is believed to have a mitochondrial etiology.

Example 1: a 72 year old male with a known intolerance to statins (due to unacceptable and persistent fatigue) took a single statin dose after abstaining for several years. The first day was uneventful, and he actually felt better than on previous days. The next day, however, he awoke with a paralyzed left arm. The paralysis was total, with no sensation or motility. This situation resolved after a couple of hours, with no tingling or other side-effects. Paralysis returned on subsequent days at irregular intervals and with increasing frequency. His upper lip then began to feel numb along with the arm paralysis, and his left leg began to feel unstable. The paralysis progressed in this way for a week, with fine finger control lost in the left hand, even when not paralyzed. Up-latching increased the symptoms, while down-latching reduced them, suggesting mitochondrial involvement. Nothing stopped the progression, however, until a single dose of 100 mg PQQ and 2 grams of AAKG was taken, which banished the paralysis. A second dose was taken 12 hours later as insurance. This second dose was 250 mg PQQ and 2 grams AAKG, and was probably not needed. No instances of paralysis occurred after either of those doses, but fine movements of the fingers of the left hand remained impaired, resulting in a nearly 100% error rate while typing. This problem slowly improved, and the rate of improvement was substantially increased by daily dosing with 200-500 mg ceramides. Within 10 days, the last of these side effects disappeared, and at that point, his typing error rate was actually lower than before the statin dose.

It is herein hypothesized that mtDNA methylation damage builds up throughout life, contributing to many other diseases of aging, but can also be an artifact of drugs or viruses. By using the PQQ/AKG treatment and the up-latchers described herein, a lifetime of mitochondrial deficiency can be quickly erased, and ATP output increased to a level not previously possible.

Example 2: A 70 year old woman used a single dose of 2 grams of AAKG and 100 mg of PQQ. Her baseline of 26 bicep reps of a 10 pound dumbbell to failure, measured just before taking the dose, increased to 46 reps when measured 2 hours later.

Returning now to FIG. 3A, the fission 326 and fusion lines 332 now merged into the new baseline 350 with a reps-to-failure that was no longer altered by fission or fusion. The multiple treatments with 100 mg PQQ at points 340, 341, 342 should have produced an mtDNA count much higher than before, but this did not translate into more ATP output as represented by reps-to-failure in new baseline 350.

The failure of ATP output to follow mtDNA count was at first a mystery. It is known that the protein component density of the IMM is very high, with approximately 70 percent protein, and thus the failure of the increased mtDNA count in the matrix to push ATP output higher was likely the result of saturation of the IMM with OXPHOS complexes. Potential signaling molecules were examined that might increase mitochondrial surface area, but it was ultimately discovered that certain carboxylic acids and their salts could function as plasticizers, creating a metastable IMM state of supersaturation. It was first discovered that lactic acid (or lactates) produced reps-to-failure above the new baseline 350, and surprisingly, this enhanced result lasted far longer than the biological life of lactate-sometimes days instead of minutes.

A new mitochondrial state was thus discovered-up-latching-which appeared to be metastable. It was found that the higher level of output could be further increased by subsequent treatments via a ratcheting effect.

Returning again to FIG. 3A, an investigation was made of the effect of lactate on ATP output beginning on day 71. Lactate is produced when the ATP requirement exceeds supply during heavy exercise, and it was first hypothesized that lactate might serve as a signaling molecule.

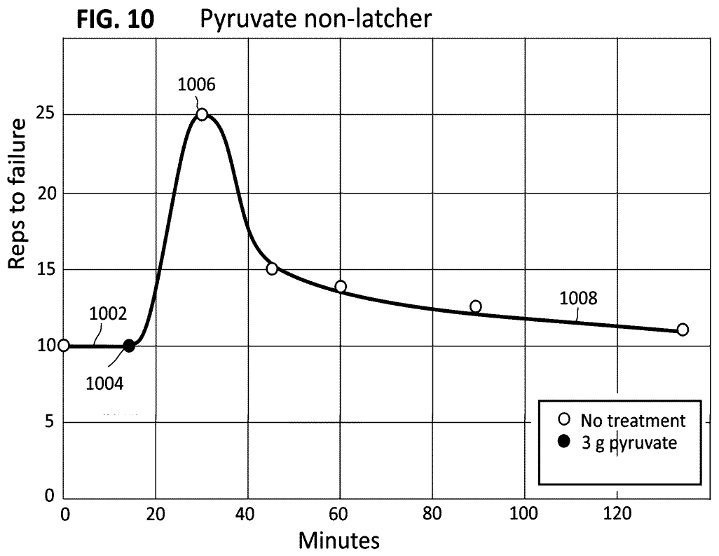

Lactate was initially dosed at points 360, 361 and 362, moving reps-to-failure above baseline 350 for the first time. More lactate dosing at points 363, 364 and 365 produced a higher plateau. Combining lactate with pyruvate and PQQ produced the highest results at points 380 and 382, but did not latch there. Pyruvate, as will be seen in FIG. 10, is a non-latcher, producing the higher result due to acting as a fuel rather than as a latcher.

The astonishing results suggested that, rather than acting as a signaling molecule, lactate likely acted as a plasticizer of the inner membrane, allowing the PQQ-enhanced concentration of OXPHOS complexes in the matrix to pack into the IMM, creating a metastable state of supersaturation and increasing ATP output. Lactate has a short biological half-life of around 20 minutes, yet the enhanced ATP output was found to last far longer, in some cases for days. It appears that ATP output can be latched at a raised level, and this up-latching can be accomplished again and again to produce a ratcheting effect. Latching is indicated by bracket 370, followed by a higher latching level indicated by bracket 372. This higher level of output can be beneficial in the treatment of many diseases, and can provide a substantial advantage in sporting performance.

It was found that most AHAs (alpha-hydroxy acids) were up-latchers. Glycolic acid at .7 g could take ATP output from 14 reps (having been knocked down by 5 g of glycerol, a down-latcher) back up to 28 reps in about 1 hour. Salts of glycolic acid (glycolates) may also be used. It was found that malic acid (in the form of magnesium malate) could take ATP output up from 10 reps (previously reduced by 3 g of glycerol and 1 g of ethyl lactate) to 21 reps in 2 hours. Likewise, 2 g of tartaric acid took ATP output from 11 reps (previously reduced by 3 g of glycerol) to 21 reps. A salt of mandelic acid at 1 g took the proxy ATP output from 14 reps (previously reduced by 3 g of glycerol) to 31 reps.

The tricarboxylic citric acid is also an AHA, but performs as a down-latcher. Since orange juice has an average citric acid concentration of 0.25 g/oz and the usual serving is 8 ounces, the dose of citric acid would be about 2 g. This dose of citric acid neutralized by bicarbonate was trialed, and was found to drop bicep curls to failure from a 24 rep baseline to 11 reps at 15 and 30 minutes. One gram each of citric acid, fumaric acid and AKG taken together produced no change from a baseline of 10 reps at 15 and 30 minutes. Just 50 mg of citric acid taken alone took reps-to-failure from 20 down to 11. A 12-oz can of cola can have 20 mg citrate or more, and 200 mL of a diet cola took reps-to-failure from 18 down to 12 reps in 20 minutes. Thus a mere glass of soda or fruit juice containing citric acid could lose a race.

All trials shown in all figures were on an empty stomach, for maximum effect.

Up-latcher molecules typically comprise one or two carboxylic end groups, but not three. Monocarboxylic and dicarboxylic acids can serve as up-latchers, while tricarboxylic acids and the esters of monocarboxylic acids and dicarboxylic acids are down-latchers,

Useful up-latchers include the carboxylic acids adipic, alpha-ketoglutaric, azelaic, fumaric, glycolic, lactic, malic, mandelic, oxaloacetic, pimelic, suberic, succinic, and tartaric acids, and preferably salts thereof, with a combined dose of at least 100 mg, more preferably 200 mg, and most preferably 500 mg or more. These are fast up-latchers. Slow up-latchers include lycopene, a carotenoid. Though slower to raise ATP, they are effective at lower doses. Up-latchers may be used singly, but preferably in multiples, and are most preferably used concurrently or subsequent to a treatment with the biogenesis promoter PQQ or derivatives thereof to increase the mtDNA count. The salt forms of carboxylic acids are preferred, and alternatively may be provided as dry acid powders to be mixed with water or other fluid that is absent down-latchers like citric acid or citrate, with sufficient carbonate, bicarbonate, oxides, or hydroxides for neutralization.

With 200 different cell types in the body, variations can be expected in uptake, and thus a mixture of up-latchers is more likely to produce full treatment coverage, and in fact multiple up-latchers, used together, showed synergistically higher bicep curls to failure than when used individually, as demonstrated in FIG. 1.

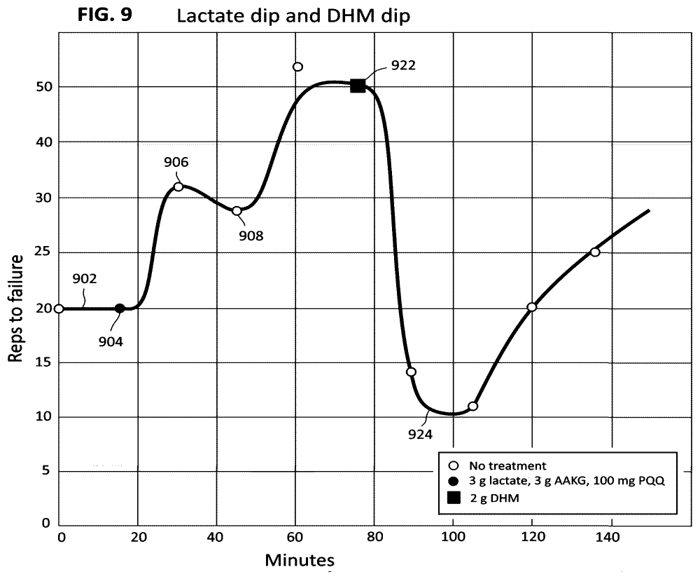

While carboxylic acids can function as up-latchers, the esters of these acids generally have the opposite effect, resulting in down-latching. Other substances also provoke down-latching or unlatching, in particular those that modify mitochondrial morphology, such as by promoting fission or fusion. For instance, B3 and derivatives thereof promote down latching due to mitochondrial fission, while DHM promotes down-latching due to mitochondrial fusion, though its behavior is more complex, as will be seen in FIG. 9. More examples are found in the non-limiting list in the table below:

TABLE 1

| | Latching | Notes |
|---|---|---|
| Lactic acid | ↑ | AHA |
| Glycolic acid | ↑ | AHA |
| Mandelic acid | ↑ | AHA |
| Malic acid | ↑ | AHA/dicarboxylic acid |
| Tartaric acid | ↑ | AHA/dicarboxylic acid |
| Fumaric acid | ↑ | Dicarboxylic acid |
| Adipic acid | ↑ | Dicarboxylic acid |
| Azelaic acid | ↑ | Dicarboxylic acid |
| Oxaloacetic acid | ↑ | Dicarboxylic acid |
| Pimelic acid | ↑ | Dicarboxylic acid |
| Suberic acid | ↑ | Dicarboxylic acid |
| Succinic acid | ↑ | Dicarboxylic acid |
| Lycopene | ↑ | Carotenoid |
| Pyruvate | — | Oxidized lactate |
| Lactate + pyruvate | — | — |
| Creatine | — | Alpha amino acid derivative |
| CoQ-10 | — | Biochemical cofactor |
| Saccharin | .— | Sugar substitute, poorly absorbed |
| Mannitol | — | Sugar alcohol, poorly absorbed |
| AHA esters | ↓ | Examples: ethyl lactate, butyl lactate |
| Dicarboxylic esters | ↓ | Example: dimethyl fumarate |
| Citric acid | ↓ | Tricarboxylic acid |
| Glycerol | ↓ | Sugar alcohol |
| Erythritol | ↓ | Sugar alcohol |
| Sorbitol | ↓ | Sugar alcohol |
| Triethanolamine | ↓ | Tertiary amine |
| Resveratrol | ↓ | Phenol |
| Phthalate ester | ↓ | Widely used as a plasticizer |
| Urolithin A | ↓ | Benzo-coumarin |

Up-latching is indicated by an up-arrow, down-latching by a down-arrow, while others producing no significant effect are indicated by a dash. Substances like creatine and pyruvate produced short term increases, but did not latch after a few hours. Plasticizers that are esters of monocarboxylic acid and dicarboxylic acids are down-latchers. Many sugar alcohols are down-latchers, depending on how well they are absorbed. Glycerol and erythritol are strong down-latchers. The tricarboxylic citric acid and salts thereof were the strongest down-latchers evaluated. Not all down-latchers are clearly plasticizers. For instance, NAD⁺ promoters such as Urolithin A appear to be down-latchers, and nitrate salts are down-latchers.

Up-latchers typically have a carboxylic (COOH) end group, while the dicarboxylic fumaric and malic acids have two such end groups. The salts of carboxylic acids with linear backbones are typically up-latchers, while their esters are typically down latchers. It is noted that an ester of fumaric acid (dimethyl fumarate) was once trialed in Germany for treating psoriasis and MS. It is suggested herein that the effectiveness was due at least in part to down-latching of the IMM and the resultant reduction of ATP and ROS output.

In down-latching, the metastable state of IMM supersaturation is lost and excess OXPHOS complexes are ejected. Thus, in one embodiment of the invention, up-latching and down-latching can be used sequentially to eject old complexes from the IMM and replace them with new complexes.

In another embodiment of the invention, up-latching can be used in conjunction with mitochondrial fusion, or in place of fusion for promoting the symmetric division of stem cells, especially when latching promoters are dosed with or before dosing with the putative UCP2 blocker C60 and/or derivatives thereof, thereby ending stem cell quiescence and resulting in SC ATP output increasing far more than without up-latching.

Down-latching achieved in the experiment of FIG. 3A is indicated by brackets 374 and 376. At point 373, a dose comprising 5 g glycerol took reps-to-failure down to baseline, and similarly at point 375, where a dose comprising 2 g triethanolamine took reps-to-failure nearly down to baseline. Furthermore, by combining down-latchers, it is possible to go substantially below baseline. After down-latching, ATP output can be rapidly increased back to the ratcheted level as occurred at point 382, which suggests that the IMM surface area was increased for at least several days after the previous latched state.

While AHAs substantially promote up-latching, one of the best up-latchers evaluated was found to be the dicarboxylic fumaric acid and its salts and isomer, while succinic acid, an alpha-omega-dicarboxylic acid, is an indirect up-latcher, converting to fumaric acid after a short delay, and thus is less preferred. Other dicarboxylic acids are good up-latchers, such as azelaic acid, and also malic acid, which is also an AHA. One gram of azelaic acid, used alone, increased reps-to-failure from 13 to 43 in 30 minutes.

It was previously hypothesized that using biogenesis during fission to reduce membrane potential to zero would preferentially expose methylated mtDNA to the PINK1/Parkin QC process, marking these mitochondria for mitophagy. Isolated mtDNA loops with the greatest methylation would have the least reserves of ATP-producing complexes during biogenesis and thus would be the most likely to be labeled for removal. Methylation would also be lost by the action of demethylase enzymes during biogenesis, a point where methyltransferase enzymes cannot restore them. However, it is now found that a sufficient dose of PQQ (or salts and/or esters) and AKG (and/or salts), can achieve this goal in as little as just one treatment by combining a high dose of a promoter for biogenesis with a promoter for demethylase. This can be combined with up-latching (either concomitated or subsequently) to produce much higher reps-to-failure, which can be ratcheted up further with multiple treatments. For removing mutated (as opposed to epimutated) mtDNA, however, fission is still required. Fission is important in diseases like Parkinson's, where aberrant ubiquitination has been reported to reduce the effectiveness of Parkin in QC, resulting in the eventual formation of Lewey bodies. For treating Parkinson's, then, fission might be combined with down-latching. For example, a combination of citrate and B3, and optionally with AKG or salts thereof.

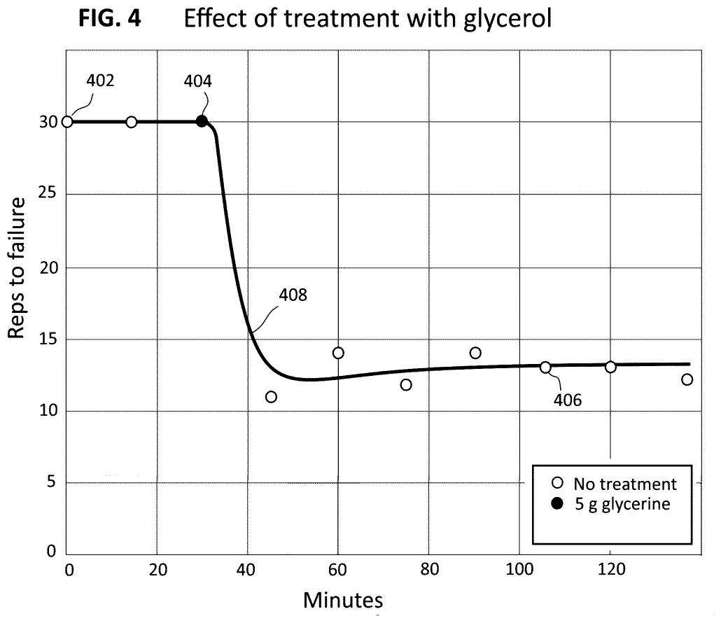

Turning now to FIG. 4, a bicep curl reps-to-failure trial began with 30 reps-to-failure at point 402, and five grams of glycerol were dosed at point 404. Reps-to-failure then fell rapidly along curve 408, stabilizing at about 13 reps, as at point 406. Other sugar alcohols have the same down-latching effect, as long as they are readily absorbed.

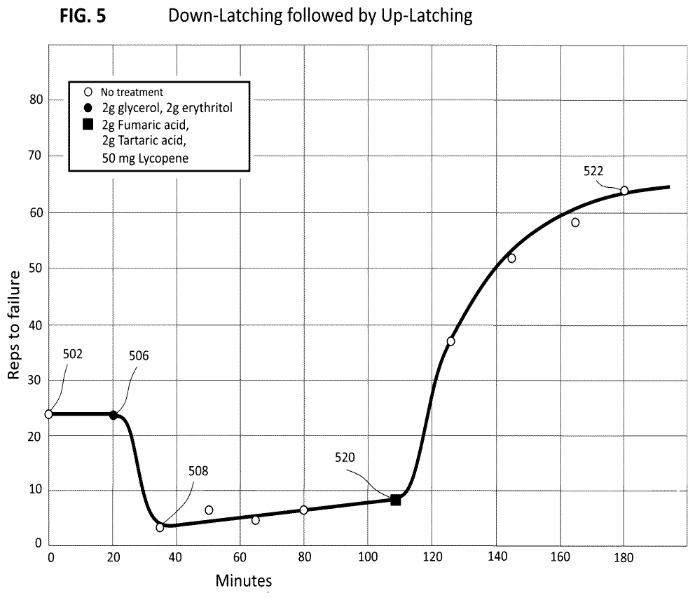

FIG. 5 is a plot of reps-to-failure that begins with a baseline of 24 reps at point 502. Treatment at point 506 consisted of a combination of down-latchers glycerol and erythritol, after which reps-to-failure declined precipitously to four reps at point 508. This slowly recovered to 9 reps at point 520. The slow recovery during down-latching was seen repeatedly, and is expected as the IMM is under-saturated. Treatment with fumaric acid, tartaric acid (both neutralized with sodium bicarbonate) and lycopene at point 520 then produced a rise over a period of 70 minutes to 64 reps at point 522.

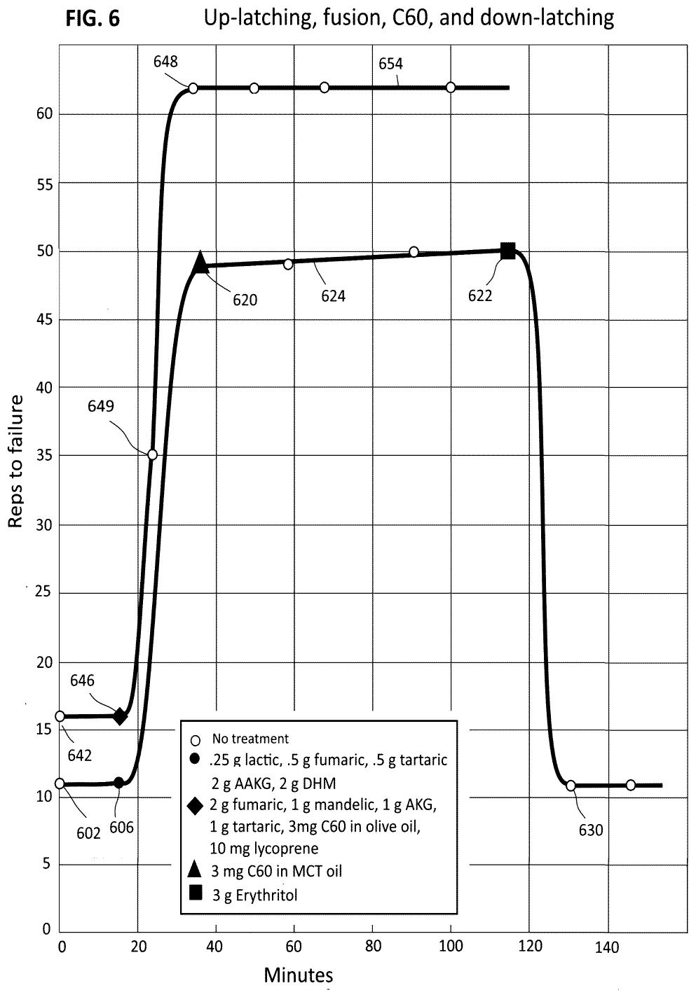

FIG. 6 shows the results of two trials using the putative UCP2 blocker C60. The first trial began at a baseline of 11 reps at point 602. At point 606, a dose was taken of 4 up-latchers and the fusion agent dihydromyricetin (DHM). In 15 minutes, reps-to-failure reached 49 at point 620, where a dose of 3 mg of C60 in MCT oil was also taken. The subsequent performance indicated by curve 624 is almost flat until point 622 where a dose of the down-latcher sugar substitute erythritol was taken, dropping the performance to the original baseline of 11 reps at point 630.

In the upper curve of FIG. 6, a combination of up-latchers and 3 mg of C60 dissolved in olive oil was dosed at point 646 on an empty stomach, with no fusion supplement. Just 7 minutes later, reps-to-failure had risen from 16 at points 642 and 646 to 35 at point 649, and in another 8 minutes to 62 reps at point 648, with subsequent performance illustrated by line 654, which is completely flat.

C60 has a very large effect on stem cells that have an estimated 10 times as many UCP2 pores as somatic cells, and was expected to add slightly to reps-to-failure for somatic cells as well, but the flattening of the peak performance was unexpected in both experiments. C60 may be used with the latching plasticizers in a unitary dose, or afterwards when the reps-to-failure are still elevated. DHM may be used with the unitary dose as well, but if used later it can result in a rapid reduction in reps, as will be seen later in FIG. 9.

Turning now to FIG. 7, the contrasting behavior of two carotenoids is shown. Lycopene was found to be an up-latcher, albeit with a relatively slow onset, while zeaxanthin is a substantially non-latcher. 100 mg of lycopene raised bicep curls to failure from a baseline of 6 reps at points 722 and 724 to 32 reps in 70 minutes after treatment at point 724, along slowly rising solid curve 728. Zeaxanthin raised bicep curls to failure from a baseline of 9 reps at points 702 and 704 to a maximum of 14 reps in 15 minutes at point 706, and then fell back as shown by the dashed curve 708. It is believed that the relatively bulky end groups of zeaxanthin prevent ready penetration into the IMM, and thus the relatively small effect on reps-to-failure.

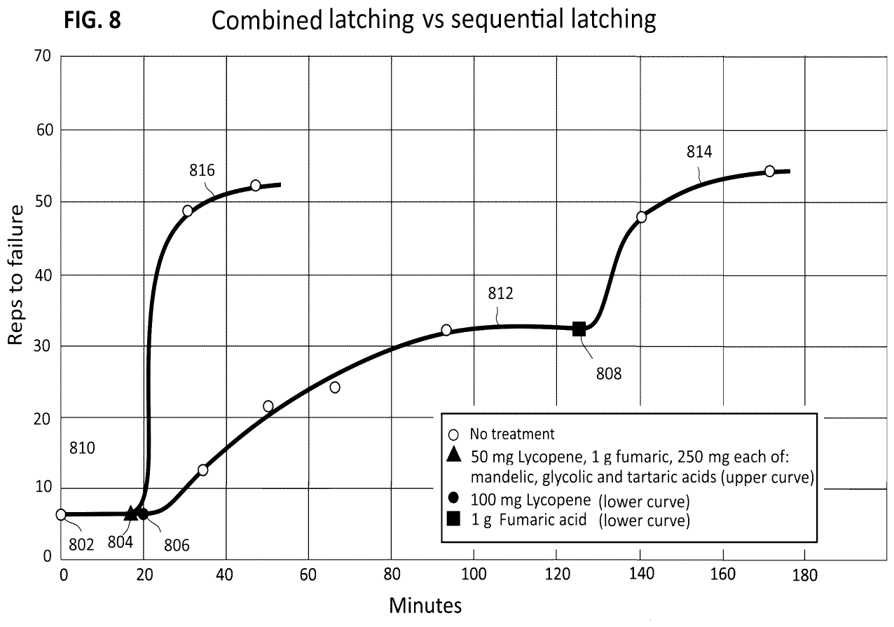

Turning now to FIG. 8, the rapidity of combined doses is compared to sequential dosing. Upper curve 816 and lower curve 812 begin at 6 reps-to-failure at point 802. The dosing of lycopene at point 806 produces a slowly rising curve 812 followed by a more rapidly increasing curve 814 by dosing neutralized fumaric acid at point 808. Ratchet curve 814 has a faster rise than curve 812, while the upper curve 816, is even faster. Curve 816 begins at point 804 where five combined latchers were dosed. The reps for the combined doses rises rapidly to a final reps-to-failure in about 30 minutes, much faster than the 140 minutes the two sequential doses took in the lower curves 812, 814. Thus it can be seen that combinations of carboxylic acids are fast up-latchers while carotenoids are relatively slow-up latchers. It can also be seen that different groups of up-latchers can be used sequentially and still get ratcheting.

Turning now to FIG. 9, the anomalous behaviors of lactate and the fusion agent DHM are shown. With a baseline 902 of 20 reps, 3 grams of lactate were dosed in combination with AAKG and PQQ at point 904, producing a first peak at point 906, then a dip at point 908, and a subsequent peak near point 922. The dip is believed to result from the conversion of lactate to pyruvate. Pyruvate is not an up-latcher, as will be seen in the next figure. Also at point 922, fusion agent DHM was dosed, which produced a large dip in curve 924, substantially below baseline, and then a recovery. Preferably, therefore, DHM should be dosed prior to or concurrently with up-latchers, rather than afterwards.

Turning now to FIG. 10, pyruvate is shown to be a non-latcher. Ten reps-to-failure at baseline 1002 are increased by dosing at point 1004 to twenty-five reps-to-failure at point 1006, where the improvement falls off quickly along descending curve 1008 to near the original baseline 1002. Pyruvic acid is derived from lactic acid via lactate dehydrogenase, and conversion of lactate to pyruvate is a possible source of the lactate dip seen in FIG. 9.

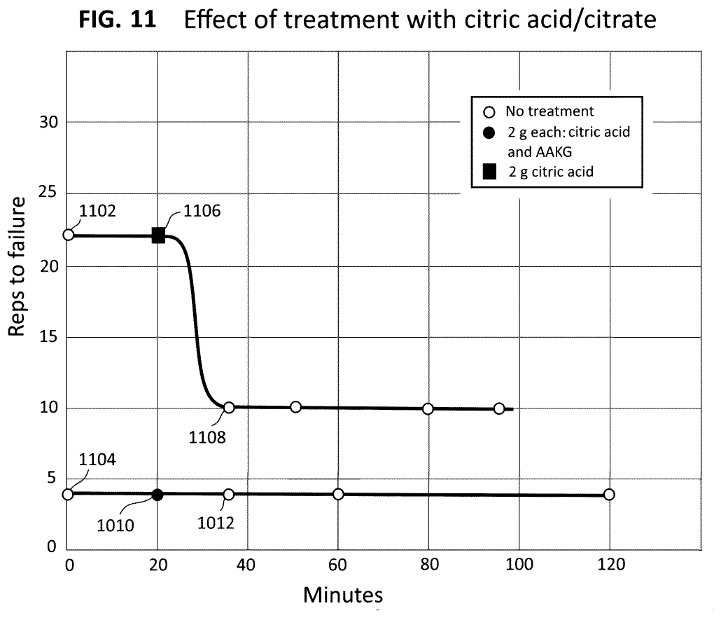

FIG. 11 shows the response of dosing neutralized citric acid from two baselines. The upper curve begins at a baseline of 22 reps established at point 1102. Two grams of neutralized citric acid were dosed at point 1106, resulting in a quick decline to 10 reps at point 1108, which continues in a perfectly flat fashion. The lower curve begins at a low baseline of 4 reps at point 1104. Two grams each of neutralized citric acid and AAKG were dosed at point 1010, which did not change 15 minutes later at point 1012, or for the next 80 minutes, though a small increase would have been expected from AAKG alone. Thus the tricarboxylic citric acid is a strong down-latcher. The half-life in the body for citrate is only about 5 minutes, so the flat line it produces in reps-to-failure is particularly impressive. And as noted previously, there are approximately 2 grams of citric acid in a glass of orange juice, which could ruin up-latching for the unwary.

### Preferred Up-Latchers:

Nutritional supplement compositions for increasing mitochondrial ATP output preferably comprise at least two different inner mitochondrial membrane up-latchers, wherein the up-latchers are salts of carboxylic acids having an unbranched backbone of 2 to 9 carbons, with a first carboxylic moiety at the proximal end, a hydroxy group at the alpha position, and/or a second carboxylic moiety at the distal end, and with an optional second hydroxy group at the alpha position relative to the distal carboxylic moiety. This latter structure is exhibited by the 4-carbon up-latcher, tartaric acid.

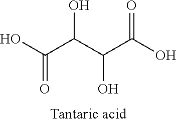

Carboxylic acids with two carboxylic end groups (dicarboxylic acids) found useful for up-latching include fumaric, oxaloacetic, malic, tartaric, and succinic acids (each with 4-carbon backbones), alpha-ketoglutaric and pimelic acids (each with 5-carbon backbones), adipic (with a 6-carbon backbone), pimelic (with a 7-carbon backbone), suberic acid (with an 8-carbon backbone), and azelaic acid (with a 9-carbon backbone).

The dicarboxylic alpha-ketoglutaric acid, while only a weak up-latcher when used alone, is synergistic with other up-latchers, and has many functions in the body as its conjugate base alpha-ketoglutarate, including as an intermediate in the citric acid cycle and a promoter of demethylase.

Carboxylic acids with a hydroxy group at the alpha position relative to a carboxyl end group (AHAs) found useful for up-latching include glycolic acid (with a 2-carbon backbone), lactic acid (with a 3-carbon backbone), tartaric acid (with a 4-carbon backbone), and mandelic acid (with a 2-carbon backbone and a benzene ring).

Malic and tartaric acids are simultaneously AHAs and dicarboxylic acids.

Fast up-latchers include salts of the following acids: adipic, alpha-ketoglutaric, azelaic, fumaric, glycolic, lactic, malic, mandelic, oxaloacetic, pimelic, suberic, succinic, and tartaric. Slow up-latchers include the following carotenoids: lycopene, phytoene and phytofluene.

Salts of carboxylic acids are preferred for up-latching, as their function is not that of acids, but of IMM plasticizers, and will interact with mitochondria as the anion of a salt in any case. A neutralizing agent or agents is preferably used with the acids, especially when the composition is supplied in powder form for mixing with water or juice. The neutralizer may supply alkali metal cations or basic amino acids such as arginine, lysine or histidine.

### Preferred Down-Latchers:

Down-latchers include tricarboxylic acids and their salts, such as citric acid and citrates, and sugar alcohols such as glycerol, sorbitol and erythritol, which may be used singly or in combination with each other and with fission. Niacin and nicotinamide are preferred for fission, and are down-latchers. The esters of carboxylic up-latchers are also down-latchers. Urolithin A (sold partially for improving athletic performance by activating mitophagy) and resveratrol have also been found to be down-latchers.

### Preferred Doses

The following are therapeutically effective doses for the above anti-aging protocols. Doses are based on an 80 kg human subject.
Fast up-latchers: Mixed AHAs, dicarboxylic acids, and/or salts thereof—1-200 mg/kg, preferably 2-100 mg/kg, and most preferably 3-50 mg/kg
Slow up-latchers: Carotenoids—0.01-20 mg/kg, preferably .02-10 mg/kg, and most preferably 0.05-5 mg/kg
Up-latcher and demethylase promoter: AKG—0.1-500 mg/kg, preferably 1-200 mg/kg, and most preferably 2-100 mg/kg
Fusion promoter: Myricetin and dihydromyricetin (DHM)—0.5-500 mg/kg, preferably 1-200 mg/kg, and most preferably 5-100 mg/kg
Fission promoter: B3—Nicotinic acid nicotinamide—1-100 mg/kg, preferably 2-50 mg/kg, and most preferably 5-20 mg/kg
Biogenesis promoter: PQQ and derivatives—50-500 mg for rapid demethylation as an attack (initial) dose and increased mtDNA count, and 2.5 mg to about 50 mg or more when used for maintenance with carboxylic treatments.
SC promoter: C60—about 1 μg to 1 mg/kg, dissolved in a biocompatible fluid, preferably food grade.

### CONCLUSION

Aging is herein seen to be promoted by methylation of mtDNA, resulting in a decline of ATP output. Methylation can be rapidly removed by simultaneously promoting mtDNA biogenesis and demethylase, with as little as a single dose. ATP output can be further increased by plasticizing the mitochondrial inner membrane (IMM) during or subsequent to mtDNA biogenesis to supersaturate the IMM with OXPHOS complexes. Latching freezes mitochondrial morphology and IMM complex density, up-latching ATP output at a higher level. A UCP2 blocker can be used with up-latching (and optionally with a mitochondrial fusion promoter) to promote symmetric stem cell division and refill stem cell niches.

The section headings used above are for organizational purposes only and are not to be construed as limiting. And although only a few exemplary embodiments of this invention have been described in detail, those skilled in the art will readily appreciate that many modifications are possible in the exemplary embodiments without materially departing from the novel teachings and advantages of this invention. Accordingly, all such modifications are intended to be included within the scope of this invention as defined in the following claims.

I claim:

1. A composition for up-latching mitochondrial energetics and reversing aging of a mammalian organism, comprising:
(a) at least one carboxylic acid selected from the group consisting of adipic, azelaic, fumaric, glycolic, lactic, malic, mandelic, oxaloacetic, pimelic, suberic, succinic, and tartaric acids, with a combined dose of at least 500 mg;
(b) at least 100 milligrams of alpha-ketoglutaric acid or salt thereof; and
(c) substantially no citric acid or salt thereof.

2. A composition as recited in claim 1, further comprising at least 50 mg of a second carboxylic acid selected from the recited group in (a).

3. A composition as recited in claim 2, further comprising at least 50 mg of a third carboxylic acid selected from the recited group in (a).

4. A composition as recited in claim 1, wherein all doses are combined for oral administration.

5. A composition as recited in claim 1, wherein one or more acid neutralizers are combined with the selected acids.

6. A composition as recited in claim 1, wherein the selected acid(s) of the group recited in (a) are pre-neutralized.

7. A composition as recited in claim 1, further comprising:
(d) 0.1 to 80 mg of the fullerene C60 or derivatives thereof, dissolved in a biocompatible fluid.

8. A composition as recited in claim 7, further comprising:
(e) a therapeutically effective dose of a mitochondrial fusion promoter.

9. A composition as recited in claim 8, wherein the fusion promoter is selected from the group consisting of stearic acid and/or source thereof; myricetin, dihydromyricetin (DHM) and other derivatives and analogs of myricetin; and sulforaphane or sources thereof.

10. A composition as recited in claim 1, further comprising at least 1 mg of a carotenoid.

11. A composition as recited in claim 10, wherein the carotenoid is selected from the group consisting of lycopene, phytoene, and phytofluene.

12. A composition as recited in claim 1, wherein doses are provided in solutions, tablets, capsules, powders, or combinations thereof.

13. A composition as recited in claim 1, wherein all doses are combined for oral administration.

14. A composition for up-latching mitochondrial energetics and reversing aging of a mammalian organism, comprising:
(a) at least two carboxylic acids selected from the group consisting of adipic, alpha-ketoglutaric, azelaic, fumaric, glycolic, lactic, malic, mandelic, oxaloacetic, pimelic, suberic, succinic, and tartaric acids, with a combined dose of at least 200 mg; and
(b) at least 20 mg of PQQ or derivative thereof.

15. A composition as recited in claim 14, wherein one or more acid neutralizers are combined with the selected acids.

16. A composition as recited in claim 14, wherein the selected acids in the group recited in (a) are pre-neutralized.

17. A method for up-latching mitochondrial energetics and reversing aging of a mammalian organism, comprising:
(a) supplying an oral dose of a biogenesis promoter; and
(b) supplying an oral dose of an up-latching plasticizer of the mitochondrial inner membrane;
whereby ATP output is increased.

18. A method as recited in claim 17, further comprising delivering 500 mg or more of alpha-ketoglutaric acid or salt thereof in (a) and/or (b).

19. A method as recited in claim 17, wherein (b) is repeated at least once.

20. A method as recited in claim 17, further comprising:
(c) down-latching ATP output by supplying an oral dose of one or more selected from the group consisting of citric acid and salts thereof; the sugar alcohols glycerol, sorbitol and erythritol; and B3.
